(19) United States
(12) Patent Application Publication
(10) Pub. No.: US 2026/0335344 A1
(43) Pub. Date: Oct. 29, 2026

(54) NOVEL FORMULATIONS AND USES

(71) Applicant: DANSTAR FERMENT AG, Zug (CH)

(72) Inventor: Marcia DA SILVA PINTO, Reinach (CH)

(21) Appl. No.: 19/109,814

(22) PCT Filed: Sep. 6, 2023
(86) PCT No.: PCT/IB2023/058814

§ 371 Date: Mar. 7, 2025

Related U.S. Application Data
(60) Provisional application No. 63/374,892, filed on Sep. 7, 2022.

(51) Int. Cl. A61K 31/7004; A61K 8/60; A61P 17/00; A61Q 19/00

(52) U.S. Cl. CPC A61K 31/7004; A61K 8/60; A61P 17/00; A61Q 19/00; A61K 2800/30

(57) ABSTRACT

The disclosure provides methods for treating or preventing inflammatory conditions of the skin and methods of controlling and maintaining skin microbiota homeostasis, comprising administering a topical formulation comprising arabinose, in particular L-arabinose, wherein the concentration of arabinose in the topical formulation is effective to promote or maintain one or more species of skin bacteria that convey a beneficial effect and/or to inhibit or maintain one or more species of skin bacteria that convey a harmful effect; as well as topical compositions of arabinose, in particular L-arabinose, useful in such methods.

### FIELD

The disclosure relates to arabinose formulations and the use of arabinose, in particular L-arabinose, to promote healthy skin microbiota and to treat inflammatory skin conditions.

### BACKGROUND

Arabinose (C₅H₁₀O₅) is an aldopentose, i.e., a monosaccharide containing five carbon atoms, which in its linear form contains an aldehyde (CHO) functional group. Arabinose exists in “D”-form or “L”-form, i.e., as D-arabinose or L-arabinose, respectively. L-Arabinose is more common than D-arabinose in nature and is the preferred form according to the present invention. L-Arabinose may exist in linear aldehyde form:

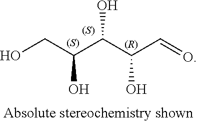

in the form of a 5-membered ring (L-arabinofuranose):

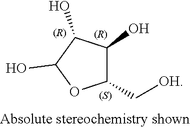

or in the form of a 6-membered ring (L-arabinopyranose):

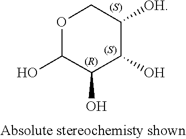

L-Arabinose occurs naturally in most plants. It has found utility as a “sugar blocker” in dietary supplements, due to its ability to inhibit absorption of sucrose by inhibiting intestinal sucrase, an enzyme that breaks down sucrose into glucose and fructose.

Arabinose (such as L-arabinose, D-arabinose, or mixtures thereof) is commercially available or can be prepared as described in the literature. L-Arabinose for example can be prepared as described in WO 2021/123004 A1. Moreover, it is well known to a person skilled in the art how to prepare the arabinose containing formulations/compositions disclosed herein.

Human skin is densely colonized with diverse and active populations of microorganisms—bacteria, fungi, viruses, and mites—which collectively form the skin microbiota. While there are many skin care products advertised as having “anti-microbial” properties, emphasizing the potential of microbes on the skin to result in disease and contamination, the skin microbiota also contributes to the protective functions of the human skin in many ways. There are extensive communications and complex interactions among bacteria, skin cells and immune cells. These interactions, in some cases, help to reinforce and repair the skin barrier, bolster the body's defenses against infection and lessen excess inflammation. Perturbations in this ecosystem can leave the skin susceptible to immune hypersensitivity disorders, such as atopic dermatitis and skin allergies, or interfere with healing in people with chronic wounds. Also, an imbalance in the skin microbiota can lead to severe skin pathologies. Since the skin microbiota is extremely important to the health of the skin, there is thus a need for better methods of controlling the skin microbiota so as to promote beneficial microbes while suppressing harmful microbes.

### SUMMARY

It has now been discovered that L-arabinose has important beneficial effects on skin microbiota, providing important and unexpected benefits in addressing a variety of conditions.

Using metagenomic sequencing, we determined the effect of various concentrations of L-arabinose on the cutaneous microbiota homeostasis. We have found that L-arabinose at concentrations of 0.5% promotes Acinetobacter baumannii, an important finding as Acinetobacter species protect against allergic sensitization and inflammation through activation of interleukin 10 (IL-10). IL-10 is a cytokine with potent anti-inflammatory property and plays an essential role in limiting host immune response to pathogens. At the same time, these concentrations of L-arabinose inhibit Micrococcus luteus. Micrococcus luteus appears to have a dual role: it prevents cutaneous membrane infection and helps DNA repair from UV damage, but at the same time it can favor initial infection from pathogenic bacteria like Staphylococcus aureus. Limiting skin inflammation would likely reduce chronic inflammation due to aging, and so reduce the effects of aging. Therefore, preventing the transversal contributor of the aging process that are the immune dysfunction in inflammatory pathways could be essential to stop the propagation of tissue deterioration due to aging. The effect of L-arabinose on the skin microbiota at concentrations of 0.5% may therefore protect against inflammation and prevent or reduce sensitive skin (and therefore prevent skin aging, also associated with chronic inflammation).

At higher concentrations of L-arabinose (3.5% and 5%), L-arabinose promotes Streptococcus mitis, a bacterium that is known to limit Staphylococcus aureus virulence implicated in atopic dermatitis and cellulitis. Moreover, at concentrations of 1% and 2% (especially at 1%), L-arabinose has the effect of maintaining the homeostasis of skin microbiota.

Accordingly, the disclosure provides, methods for promoting healthy skin microbiota comprising topically administering an effective amount of arabinose, in particular L-arabinose, to the skin of a subject, such as a mammalian subject, in particular a human subject, in need thereof, e.g., methods for protecting against or reducing skin inflammation (including allergic sensitization, chronic inflammation associated with aging, atopic dermatitis and cellulitis) by promoting skin bacteria that convey a beneficial effect and/or inhibiting bacteria that convey a harmful effect, including infectious pathogens, e.g., Staphylococcus aureus, and methods for controlling and maintaining skin microbiota homeostasis.

The disclosure also provides topical compositions comprising arabinose, in particular L-arabinose, and arabinose, in particular L-arabinose, for use in methods as described.

### DRAWINGS

FIG. 1 depicts the relative quantity of bacteria in the non-treated and non-stressed (NT-NS) control condition after sequencing.

FIG. 2 depicts the relative quantity of Acinetobacter baumannii at different concentrations of L-arabinose.

FIG. 3 depicts the relative quantity of Micrococcus luteus at different concentrations of L-arabinose.

FIG. 4 depicts the relative quantity of Staphylococcus epidermidis at different concentrations of L-arabinose.

FIG. 5 depicts the relative quantity of Streptococcus mitis at different concentrations of L-arabinose.

FIG. 6 depicts the relative quantity of bacteria in the skin microbiota mix at different concentrations of L-arabinose.

### DETAILED DESCRIPTION

In a first embodiment, the disclosure provides a method (Method 1) for treating or preventing an inflammatory condition of the skin in a subject, such as a mammalian subject, in particular a human subject, in need thereof, comprising administering to the skin of the subject a topical formulation comprising arabinose, in particular L-arabinose, wherein the concentration of arabinose in the topical formulation is effective to promote one or more (especially one) or to maintain one or more species of skin bacteria that convey a beneficial effect and/or to inhibit one or more (especially one or two) or to maintain one or more species of skin bacteria that convey a harmful effect. For example, Method 1 includes the following methods:
1.1 Method 1 wherein the topical formulation is effective to promote one or more (especially one) species of skin bacteria that convey a beneficial effect and/or to inhibit one or more (especially one or two) skin bacteria that convey a harmful effect.
1.2 Any foregoing Method wherein the topical formulation is effective to maintain one or more species of skin bacteria that convey a beneficial effect and/or to maintain one or more species of skin bacteria that convey a harmful effect.
1.3 Any foregoing Method wherein the concentration of arabinose in the topical formulation is 0.1% to 10%.
1.4 Any foregoing Method wherein the concentration of arabinose in the topical formulation is 0.1% to 5%, 0.5% to 5%, 0.5% to 3%, 0.1% to 1%, 0.5% to 1%, 1% to 2%, 2.5% to 5%, 3% to 5%, or 3.5% to 5%.
1.5 Any foregoing Method wherein the concentration of arabinose in the topical formulation is about 0.5%, about 1%, about 2%, about 2.5%, about 3%, about 3.5%, about 4%, or about 5%.
1.6 Any foregoing Method for preventing allergic sensitization.
1.7 Any foregoing Method for treating chronic inflammation of the skin associated with aging.
1.8 Any foregoing Method for treating atopic dermatitis.
1.9 Any foregoing Method for treating cellulitis.
1.10 Any foregoing Method wherein the condition to be treated or prevented is characterized by increased levels of IL-10.
1.11 Any foregoing Method wherein the bacteria that conveys a beneficial effect comprises an Acinetobacter species, in particular Acinetobacter baumannii.
1.12 Any foregoing Method wherein the subject exhibits an increase in the level of an Acinetobacter species, in particular Acinetobacter baumannii, on the skin following administration of the topical formulation.
1.13 Any foregoing Method wherein the bacteria that conveys a beneficial effect comprises Streptococcus mitis.
1.14 Any foregoing Method wherein the subject exhibits an increase in the level of Streptococcus mitis on the skin following administration of the topical formulation.
1.15 Any foregoing Method wherein the bacteria that conveys a harmful effect comprises Staphylococcus aureus.
1.16 Any foregoing Method wherein the subject exhibits a reduction in the level of Staphylococcus aureus on the skin following administration of the topical formulation.
1.17 Any foregoing Method wherein the bacteria that conveys a harmful effect comprises Micrococcus luteus.
1.18 Any foregoing Method wherein the subject exhibits a reduction in the level of Micrococcus luteus on the skin following administration of the topical formulation.
1.19 Any foregoing Method for treating chronic skin inflammation associated with aging wherein the concentration of arabinose in the topical formulation is 0.1%-1%, especially about 0.5%.
1.20 Any foregoing Method for treating atopic dermatitis wherein the concentration of arabinose in the topical formulation is 3%-5%, especially 3.5%-5%.
1.21 Any foregoing Method wherein the topical formulation is applied to the skin once or twice daily.
1.22 Any foregoing Method wherein the duration of administration is at least one week, e.g., at least one month.
1.23 Any foregoing Method wherein the formulation is free of saccharides other than arabinose.
1.24 Any foregoing Method wherein the arabinose is L-arabinose.
1.25 Any foregoing Method wherein the topical formulation is in the form of a cream, lotion, spray, ointment, serum, lip balm, make-up, soap, shower gel, shampoo, hair conditioner, or hair mask.

The disclosure further provides arabinose, in particular L-arabinose, for use in treating or preventing an inflammatory condition of the skin in a subject, such as a mammalian subject, in particular a human subject, in need thereof by administering to the skin of the subject a topical formulation comprising arabinose, in particular L-arabinose, wherein the concentration of arabinose in the topical formulation is effective to promote one or more (especially one) or to maintain one or more species of skin bacteria that convey a beneficial effect and to inhibit one or more (especially one or two) or to maintain one or more species of skin bacteria that convey a harmful effect, e.g., for use in a method according to any one of above Methods 1.1-1.25.

If stated herein that a species of skin bacteria is promoted, it means that the relative quantity of that species of bacteria is increased compared to the total quantity of skin bacteria. If, on the other hand, it is stated that a species of skin bacteria is inhibited, it means that the relative quantity of that species of bacteria is decreased compared to the total quantity of skin bacteria.

If stated herein that a species of skin bacteria is maintained, it means that the relative quantity of that species of bacteria is maintained compared to the total quantity of skin bacteria.

Unless explicitly stated otherwise, percentages refer to percentages by weight (wt %).

The term “about” placed before a numerical value “X” refers in the current application to an interval extending from X minus 30% of X to X plus 30% of X, preferably to an interval extending from X minus 10% of X to X plus 10% of X.

The disclosure also provides a method (Method 2) of controlling and maintaining skin microbiota homeostasis, comprising administering to the skin of a subject, such as a mammalian subject, in particular a human subject, a topical formulation comprising arabinose, in particular L-arabinose, wherein the concentration of arabinose in the topical formulation is effective to promote one or more (especially one) or to maintain one or more species of skin bacteria that convey a beneficial effect and/or to inhibit one or more (especially one or two) or to maintain one or more species of skin bacteria that convey a harmful effect. For example, Method 2 includes the flowing methods:
2.1 Method 2 wherein the topical formulation is effective to promote one or more (especially one) species of skin bacteria that convey a beneficial effect and/or to inhibit one or more (especially one or two) skin bacteria that convey a harmful effect.
2.2 Any foregoing Method wherein the topical formulation is effective to maintain one or more species of skin bacteria that convey a beneficial effect and/or to maintain one or more species of skin bacteria that convey a harmful effect.
2.3 Any foregoing Method wherein the concentration of arabinose in the topical formulation is 0.1% to 10%.
2.4 Any foregoing Method wherein the concentration of arabinose in the topical formulation is 0.1% to 5%, 0.5% to 5%, 0.5% to 3%, 0.1% to 1%, 0.5% to 1%, 1% to 2%, 2.5% to 5%, 3% to 5%, or 3.5% to 5%.
2.5 Any foregoing Method wherein the concentration of arabinose in the topical formulation is about 0.5%, about 1%, about 2%, about 2.5%, about 3%, about 3.5%, about 4%, or about 5%.
2.6 Any foregoing Method wherein the bacteria that conveys a beneficial effect comprises an Acinetobacter species, in particular Acinetobacter baumannii.
2.7 Any foregoing Method wherein the subject exhibits an increase in the level of an Acinetobacter species, in particular Acinetobacter baumannii, on the skin following administration of the topical formulation.
2.8 Any foregoing Method wherein the bacteria that conveys a beneficial effect comprises Streptococcus mitis.
2.9 Any foregoing Method wherein the subject exhibits an increase in the level of Streptococcus mitis on the skin following administration of the topical formulation.
2.10 Any foregoing Method wherein the bacteria that conveys a harmful effect comprises Staphylococcus aureus.
2.11 Any foregoing Method wherein the subject exhibits a reduction in the level of Staphylococcus aureus on the skin following administration of the topical formulation.
2.12 Any foregoing Method wherein the bacteria that conveys a harmful effect comprises Micrococcus luteus.
2.13 Any foregoing Method wherein the subject exhibits a reduction in the level of Micrococcus luteus on the skin following administration of the topical formulation.
2.14 Method 2 for maintaining skin microbiota homeostasis wherein the concentration of arabinose in the topical formulation is 1% to 2%, such as about 1% or about 2%, in particular about 1%.
2.15 Any foregoing Method wherein the topical formulation is applied to the skin once or twice daily.
2.16 Any foregoing Method wherein the duration of administration is at least one week, e.g., at least one month.
2.17 Any foregoing Method wherein the formulation is free of saccharides other than arabinose.
2.18 Any foregoing Method wherein the arabinose is L-arabinose.
2.19 Any foregoing Method wherein the topical formulation is in the form of a cream, lotion, spray, ointment, serum, lip balm, make-up, soap, shower gel, shampoo, hair conditioner, or hair mask.

The disclosure further provides a topical pharmaceutical or cosmetic composition (Composition 1) comprising arabinose, in particular L-arabinose, wherein the concentration of arabinose in the topical composition is 0.1% to 10%. For example, Composition 1 includes the following compositions:
3.1 Composition 1 wherein the concentration of arabinose is 0.1% to 5%, 0.5% to 5%, 0.5% to 3%, 0.1% to 1%, 0.5% to 1%, 1% to 2%, 2.5% to 5%, 3% to 5%, or 3.5% to 5%.
3.2 Any foregoing Composition wherein the concentration of arabinose is about 0.5%, about 10%, about 2%, about 2.5%, about 3%, about 3.5%, about 4%, or about 5%.
3.3 Any foregoing Composition which is effective to promote an Acinetobacter species, in particular Acinetobacter baumannii, on the human skin.
3.4 Any foregoing Composition which is effective to promote Streptococcus mitis on the human skin.
3.5 Any foregoing Composition which is effective to inhibit Staphylococcus aureus on the human skin.
3.6 Any foregoing Composition which is effective to inhibit Micrococcus luteus on the human skin.
3.7 Any foregoing Composition which is in the form of a cream, lotion, spray, ointment, serum, lip balm, make-up, soap, shower gel, shampoo, hair conditioner, or hair mask.
3.8 Any foregoing Composition which further comprises a fragrance.
3.9 Any foregoing Composition which is free of saccharides other than arabinose.
3.10 Any foregoing Composition wherein the arabinose is L-arabinose.
3.11 Any foregoing Composition for use in any one of above Methods 1—1.25 or Methods 2—2.19.

### Example: Evaluation of the Effect of L-Arabinose on Growth of Common Skin Bacteria

The objective of the experiments is to evaluate the effect of L-arabinose at different concentrations on a bacterial pool mimicking the skin microbiota by quantification of the bacterial mix using metagenomic sequencing. Five different types of bacteria are obtained from ATCC®: Micrococcus luteus (Cat. No. 4698), Corynebacterium striatum (Cat. No. BAA-1293), Staphylococcus epidermidis (Cat. No. 12228), Acinetobacter baumannii (Cat. No. 19606) and Streptococcus mitis (Cat. No. NCIMB-13770). The bacteria are common skin bacteria, selected to roughly mimic the skin microbiota. The bacteria are grown on specific media, either solid media (Brain Heart Infusion (BHI) Agar from Condalab (Cat. No. 1048.00) or Trypticase Soy (TS) Agar from Condalab (Cat. No. 1068.00)) or on liquid media (either Brain Heart Infusion Broth from Condalab (Cat. No. 1400.00) or Trypticase Soy Broth from Condalab (Cat. No. 1224.00)). The bacteria culture is performed according to the ATCC instructions and adapted to the specific conditions of each bacterium: all bacteria are grown in BHI medium at 37° C. in an aerobic environment shaking at 120 rpm, except for Streptococcus mitis which requires an anaerobic environment, so without shaking and with paraffin oil from Sigma-Aldrich (Cat. No. 185812) above the medium to cut off contact with air.

Growth kinetics are carried out for each bacterium to determine their different growth phases and in particular the time needed to reach the exponential phase. Aerobically growing bacteria are plated on solid medium and then a colony is inoculated into 10 mL of liquid medium. An OD at 600 nm is then taken at regular time intervals from T0, the inoculation time, to obtain a growth curve until reaching the stationary phase. Anaerobically growing bacteria are directly inoculated at 500 μL into 9.5 mL of medium.

Bacteria are treated during 6 hours with L-arabinose, diluted in culture medium. L-Arabinose is first resuspended in culture medium at a concentration of 5%. The following dilutions are directly realized in culture medium at the following concentrations: 3.5%, 2%, 1% and 0.5%.

Each bacterium from the mix is initially grown independently. Once the exponential phase starts, Corynebacterium striatum, Micrococcus luteus and Staphylococcus epidermidis are mixed and grown in the presence or not of the L-arabinose at different concentrations for 6 hours, before reaching the stationary phase. Acinetobacter baumannii and Streptococcus mitis are grown independently with the L-arabinose due to their specific nature (Biosafety Level 2 (BSL-2) bacteria and anaerobic growth for Streptococcus mitis) and pulled in the final mix. Then, the mix is centrifugated and sent to sequencing. Bacterial DNA is isolated, sequenced, and 16S rRNA sequences are matched to taxonomic databases.

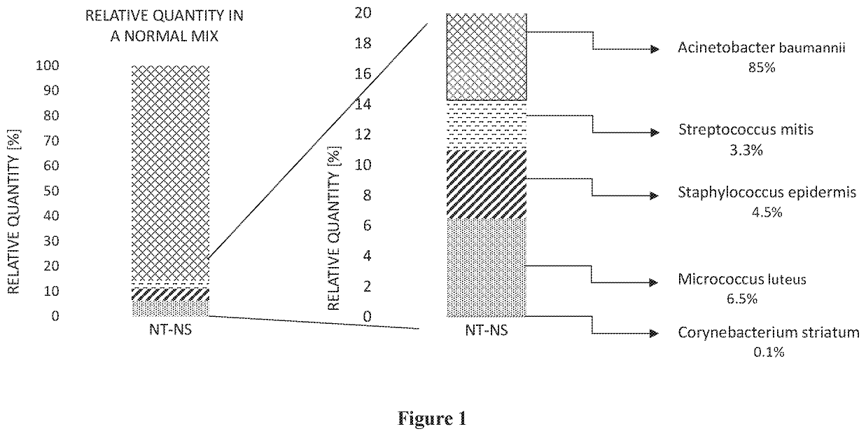

As shown in FIG. 1, all bacteria, with the exception of Corynebacterium striatum, are able to grow in the mix. FIG. 1 shows the relative quantity of bacteria in the non-treated and non-stressed (NT-NS) control condition after sequencing. Relative bacteria abundance is detected in the NT-NS control sample after metagenomic sequencing. The bacteria seeded in the mix are Micrococcus luteus, Corynebacterium striatum, Staphylococcus epidermidis. Acinetobacter baumannii, and Streptococcus mitis. All the Operational Taxonomic Units (OTUs) detected are sorted by genera to be able to differentiate all the bacteria present.

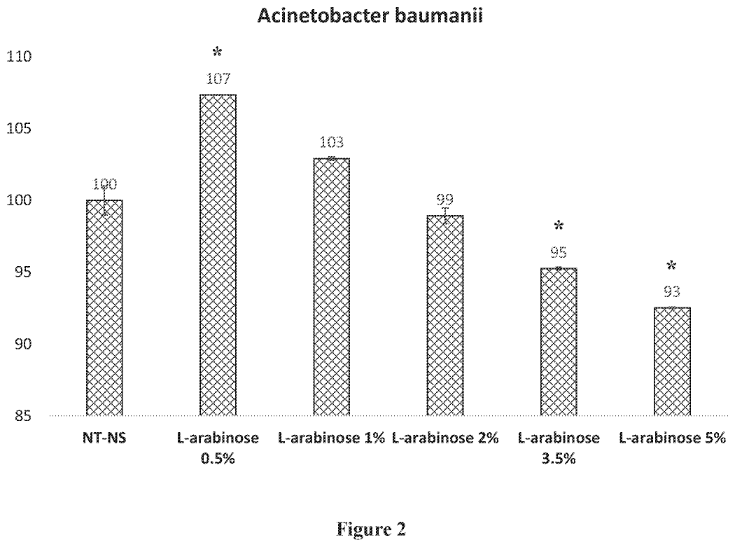

FIG. 2 depicts the relative quantity of Acinetobacter baumannii at different concentrations of L-arabinose. Relative Acinetobacter baumannii abundance is compared in all samples after metagenomic sequencing. Bacteria are treated with L-arabinose at 5 concentrations: 0.5%, 1%, 2%, 3.5% and 5%. Only the OTU identifying the Acinetobacter baumannii genera is represented.

The statistical analysis for FIGS. 2-5 is performed using two-tailed unpaired T-test vs NT-NS (*p-value<0.05, **p-value<0.01, ***p-value<0.001).

FIG. 2 shows that L-arabinose has a dose-dependent effect on Acinetobacter baumannii. It increases by 7% when using 0.5% of L-arabinose and statistically decreases up to 7% at 5% of L-arabinose compared to the NT-NS control. The relative quantity of Acinetobacter baumannii is the same than the control when using 1% or 2% of L-arabinose.

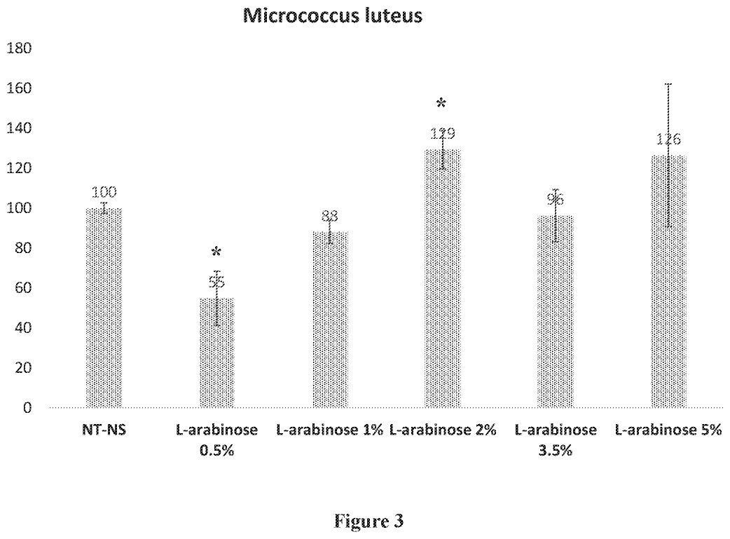

FIG. 3 depicts the relative quantity of Micrococcus luteus at different concentrations of L-arabinose. Relative Micrococcus luteus abundance is compared in all samples after metagenomic sequencing. Bacteria are treated with L-arabinose at 5 concentrations: 0.5%, 1%, 2%, 3.5% and 5%. Only the OTU identifying the Micrococcus luteus genera is represented.

As shown in FIG. 3, 0.5% of L-arabinose in the mix statistically decreases the relative quantity of Micrococcus luteus compared to the control condition. At 2% of L-arabinose there is a statistically significant increase of Micrococcus luteus. At 1%, 3.5% or 5% of L-arabinose, there is no statistically significant change in the relative quantity of Micrococcus luteus.

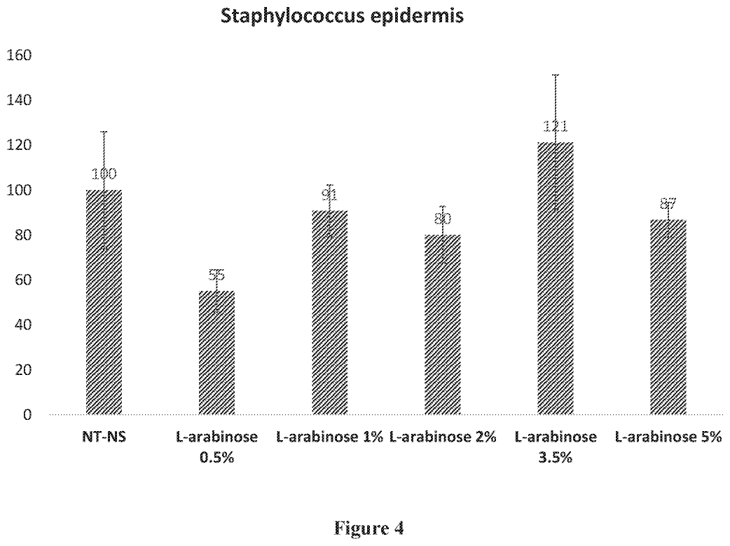

FIG. 4 depicts the relative quantity of Staphylococcus epidermidis at different concentrations of L-arabinose. Relative Staphylococcus epidermidis abundance is compared in all samples after metagenomic sequencing. Bacteria are treated with L-arabinose at 5 concentrations: 0.5%, 1%, 2%, 3.5% and 5%. Only the OTU identifying the Staphylococcus epidermidis genera is represented.

FIG. 4 shows a tendency to decrease Staphylococcus epidermidis' relative quantity after treatment with 0.5% of L-arabinose, compared to control conditions. At other concentrations there is no statistical differences in relative quantity of Staphylococcus epidermidis.

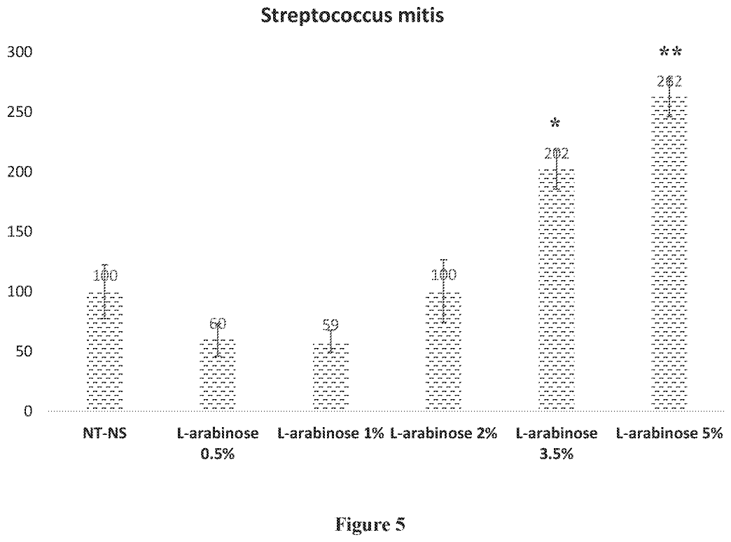

FIG. 5 depicts the relative quantity of Streptococcus mitis at different concentrations of L-arabinose. Relative Streptococcus mitis abundance is compared in all samples after metagenomic sequencing. Bacteria are treated with L-arabinose at 5 concentrations: 0.5%, 1%, 2%, 3.5% and 5%. Only the OTU identifying the Streptococcus mitis genera is represented.

As shown in FIG. 5, L-arabinose has a dose-dependent effect on Streptococcus mitis. The L-arabinose tends to decrease Streptococcus mitis' relative quantity at 0.5% and 1% and statistically increases Streptococcus mitis' relative quantity more than 2 times at 3.5% and 5%. The relative quantity of Streptococcus mitis is the same than the control when using 2% of L-arabinose.

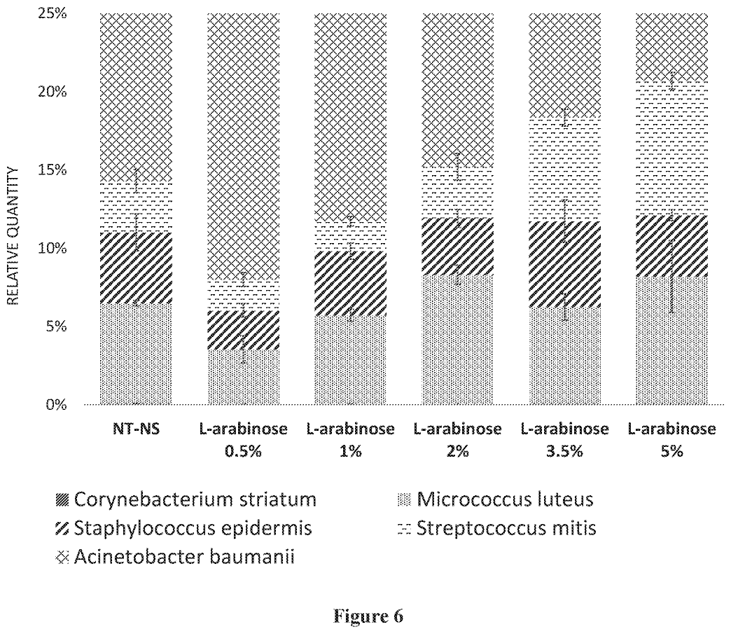

FIG. 6 depicts the relative quantity of bacteria in the skin microbiota mix at different concentrations of L-arabinose. Relative bacteria abundance is detected in all samples after metagenomic sequencing. The bacteria seeded in the mix are Micrococcus luteus, Corynebacterium striatum, Staphylococcus epidermidis, Acinetobacter baumannii and Streptococcus mitis. Bacteria are treated with L-arabinose at 5 concentrations: 0.5%, 1%, 2%, 3.5% and 5%. All the OTUs detected are sorted by genera to be able to differentiate all the bacteria present in every sample.

Looking in the whole extract, FIG. 6 shows that Acinetobacter baumannii is outgrowing the other bacteria in the mix with 0.5% of L-arabinose compared to the other bacteria. At 3.5% and 5% of L-arabinose, Streptococcus mitis is fast growing at the expense of Acinetobacter baumannii. Finally, the bacterial mix containing 1% or 2% of L-arabinose are safekeeping the homeostasis of the skin microbiota. There is no significant difference compared to the NT-NS control.

1. A method for treating or preventing an inflammatory condition of the skin in a subject in need thereof, comprising administering to the skin of the subject a topical formulation comprising arabinose, wherein the concentration of arabinose in the topical formulation is effective to promote or maintain one or more species of skin bacteria that convey a beneficial effect and/or to inhibit or maintain one or more species of skin bacteria that convey a harmful effect.

2. The method of claim 1 wherein the condition to be prevented or treated is allergic sensitization, chronic inflammation of the skin associated with aging, atopic dermatitis, or cellulitis.

3. A method of controlling and maintaining skin microbiota homeostasis, comprising administering to the skin of a subject a topical formulation comprising arabinose, wherein the concentration of arabinose in the topical formulation is effective to promote or maintain one or more species of skin bacteria that convey a beneficial effect and/or to inhibit or maintain one or more species of skin bacteria that convey a harmful effect.

4. The method of claim 1, wherein the bacteria that conveys a beneficial effect comprises an Acinetobacter species or Streptococcus mitis.

5. The method of claim 1, wherein the bacteria that conveys a harmful effect comprises Staphylococcus aureus or Micrococcus luteus.

6. The method of claim 1, wherein the concentration of arabinose in the topical formulation is 0.1% to 10%.

7. The method of claim 1, wherein the concentration of arabinose in the topical formulation is 0.1% to 5%, 0.5% to 5%, 0.5% to 3%, 0.1% to 1%, 0.5% to 1%, 1% to 2%, 2.5% to 5%, 3% to 5%, or 3.5% to 5%.

8. The method of claim 1 for treating chronic skin inflammation associated with aging wherein the concentration of arabinose in the topical formulation is 0.1%-1%.

9. The method of claim 1 for treating atopic dermatitis wherein the concentration of arabinose in the topical formulation is 3%-5%.

10. The method of claim 1, wherein the formulation is free of saccharides other than arabinose.

11. The method of claim 1, wherein the arabinose is L-arabinose.

12. A topical pharmaceutical or cosmetic composition comprising arabinose, wherein the concentration of arabinose in the topical composition is 0.1% to 10%.

13. The topical composition of claim 12 wherein the concentration of arabinose is 0.1% to 5%, 0.5% to 5%, 0.5% to 3%, 0.1% to 1%, 0.5% to 1%, 1% to 2%, 2.5% to 5%, 3% to 5%, or 3.5% to 5%.

14. The topical composition of claim 12 which is free of saccharides other than arabinose.

15. The topical composition of claim 12, wherein the arabinose is L-arabinose.

16. The method of claim 3, wherein the bacteria that conveys a beneficial effect comprises an Acinetobacter species or Streptococcus mitis.

17. The method of claim 3, wherein the bacteria that conveys a harmful effect comprises Staphylococcus aureus or Micrococcus luteus.

18. The method of claim 3, wherein the concentration of arabinose in the topical formulation is 0.1% to 10%.

19. The method of claim 3, wherein the concentration of arabinose in the topical formulation is 0.1% to 5%, 0.5% to 5%, 0.5% to 3%, 0.1% to 1%, 0.5% to 1%, 1% to 2%, 2.5% to 5%, 3% to 5%, or 3.5% to 5%.

20. The method of claim 3, wherein the formulation is free of saccharides other than arabinose.

21. The method of claim 3, wherein the arabinose is L-arabinose.
